(19) United States
(12) Patent Application Publication
(10) Pub. No.: US 2004/0167518 A1
(43) Pub. Date: Aug. 26, 2004

(54) Radiolucent frame element for external bone fixators

(76) Inventor: Hector Mark Estrada JR., Austin, TX (US)

Correspondence Address: COX & SMITH INCORPORATED, 112 EAST PECAN STREET, SUITE 1800, SAN ANTONIO, TX, 78205-1521, US

(21) Appl. No.: 10/618,253

(22) Filed: Jul. 11, 2003

Related U.S. Application Data
(60) Provisional application No. 60/414,528, filed on Jul. 12, 2002.

(51) Int. Cl. A61B 17/56

(52) U.S. Cl. 606/56

(57) ABSTRACT

An annular or arcuate frame element made of entirely of substantially radiolucent body material is provided for an external fixation device for bones. In particular, an annular or arcuate frame of substantially radiolucent body material embeds two beryllium rings. Such frames may be interchanged with the non-radiolucent annular or arcuate frames of various external fixators.

### 1. CROSS-REFERENCE TO RELATED APPLICATIONS

This application claims the benefit of the filing date of U.S. provisional patent application No. 60/414,528, filed Jul. 12, 2002, and entitled “Radiolucent Frame Element For External Long Bone Fixators,” the contents of which are hereby incorporated by reference.

### BACKGROUND OF THE INVENTION

2. Field of the Invention

The present invention relates generally to an external fixation apparatus for use in osteosynthesis and osteoplasty, particularly involving diaphyseal bone. More specifically, the present invention relates to a radiolucent component for an external fixation apparatus.

3. Background of the Invention

External fixation of bone is a well-known means of treating bone trauma and correcting deformities. Various fixation devices, or fixators, are used to support and align bone fragments in a relatively fixed relationship during regeneration or deformity correction. Such devices include the Sheffield fixator, manufactured by Orthofix, Srl, of Italy, the Ilizarov Fixator described in U.S. Pat. Nos. 4,615,338 and 4,978,347, and the fixator described in U.S. Pat. No. 4,450,834 to Fischer, each of which may utilize annular or arcuate frame segments interconnected by adjustable rods, and established around a bone by means of transfixing and non-transfixing wires and pins.

Typically, an annular, or, alternatively, an arcuate frame segment is established around either metaphyseal or diaphyseal bone by multiple transfixing wires to provide variable elastic support to the bone during loading. A second such frame is established around diaphyseal bone by either another set of transfixing wires or by non-transfixing pins. The frames are typically connected to each other threaded rods that may be adjusted so as to urge the frames either toward or away from each other into a desired relationship.

External fixation devices may be used to stabilize a bone fracture to permit bone regeneration. For this purpose, wires or screws are affixed to various bone fragments, and are further mounted to annular or arcuate frames and adjusted so as to place the bone segments in desired alignment.

Another use for external fixation devices is for distraction of diaphyseal bone for such purposes as increasing its length. It is known that bone has piezo-electric properties; that is, stresses to bone cause small electrical charges that promote bone growth. Accordingly, diaphyseal bone may be circumferentially scored and annular frames established around the bone on each side of the score by screws or wires or both. Rods connecting the frame elements are adjusted so as to provide tensile stress along the long axis of the bone. Piezo-electric stimulation of bone growth at the score site causes lengthening of the bone over a period of time.

During both installation and use of the fixator, the placement of pins and wires and the progress of bone regeneration are typically revealed by x-radiographs. Known fixator devices, however, do not include radiolucent (x-ray transparent) components. The non-radiolucent components of known fixator devices, such as the annular or arcuate frame segments, pins, wires, and connecting rods, hinder a proper view of the bone, and require that the bone be viewed from multiple and inconvenient angles. Thus, assessing pin and wire placement, as well as bone alignment and regeneration, is unnecessarily complicated by obstructing fixator components.

### SUMMARY OF THE INVENTION

It is an object of the present invention to provide an annular or arcuate fixator frame comprised of radiolucent body materials.

It is another object of the present invention to provide an annular or arcuate fixator frame comprised of radiolucent, autoclavable polycarbonate.

It is a further object of the present invention to provide an annular or arcuate fixator frame embedding stiffening rings comprised of radiolucent beryllium.

It is yet another object of the present invention to provide an annular or arcuate fixator frame that is both light weight and rigid.

Another object of the present invention is to provide an annular fixator or arcuate frame that is chemically inert with respect to the human body and commonly-encountered household substances, e.g., mild acids, alcohols and bases, such as common cleansers, hygienic and medical products, and food substances.

It is a further object of the present invention to provide an annular or arcuate fixator frame sufficiently versatile that it may be interchanged with non-radiolucent annular frames of common external fixation devices, such as those various fixators disclosed in, for example, U.S. Pat. Nos. 4,615,338, 4,450,834, 4,006,740, 4,365,624, 4,978,347 and 5,067,954.

An additional object of the present invention is to provide an annular or arcuate fixator frame to which a variety of wire- and pin-securing devices may be attached.

These and other objects and advantages will become apparent from a consideration of the accompanying drawings and ensuing description.

### BRIEF DESCRIPTION OF THE DRAWINGS

FIG. 1 is a plan view of an annular frame of the invention having multiple apertures.

FIG. 2 is a detailed cross-sectional elevation of an annular frame of the invention, shown in FIG. 1, having embedded stiffening rings.

FIG. 3 is a cross-sectional plan view of an annular frame of the invention depicting the relative position of each stiffening ring.

FIG. 4 is a perspective view generally depicting a typical installed fixator device having radiolucent annular frame components.

FIG. 5 is a plan view of an arcuate frame of the invention having multiple apertures.

### DETAILED DESCRIPTION OF THE PREFERRED EMBODIMENT

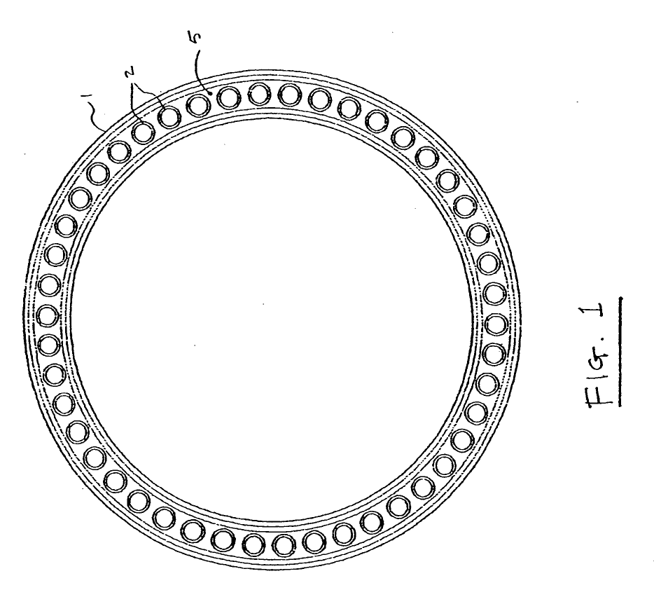

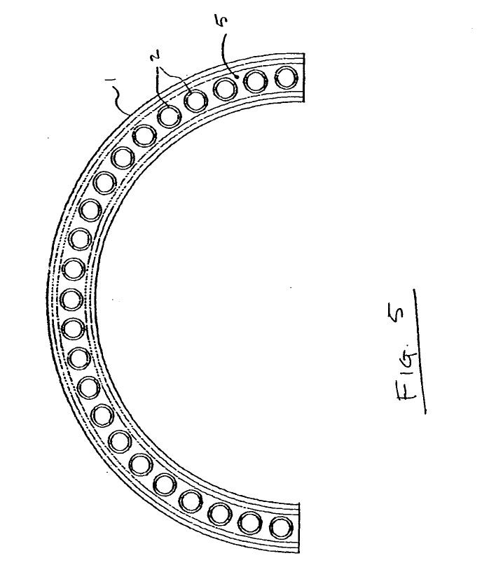

An apparatus for external bone fixation commonly includes an annular, or ring-shaped, frame 1 upon which known fixator components such as connector rods 6, pin clamps (not shown), wires 7, wire-tensioning carriages 8 and other such hardware may be mounted. This annular frame 1 will now be described in greater detail with reference to FIGS. 1-4. Those skilled in the art will appreciate that the frame 1 may be arcuate, or arc-shaped, rather than annular, as shown in FIG. 5.

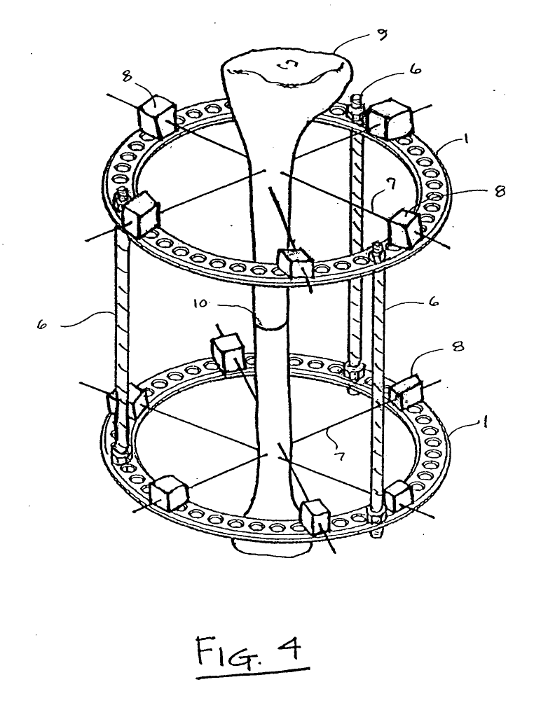

As may be seen in FIG. 1, the fixator frame 1 of one embodiment is annular, or ring-shaped, and has a generally constant thickness “t”. Preferably, the body of the frame 1 is comprised of radiolucent, autoclavable polycarbonate. Of course, those skilled in the art will recognize that the body of the frame 1 may be comprised of other types of radiolucent material, such as carbon fiber. A plurality of apertures 2 through the frame are preferably provided for rapid mounting of connector rods 6, wires 7 and wire-tensioning carriages 8, as in FIG. 4.

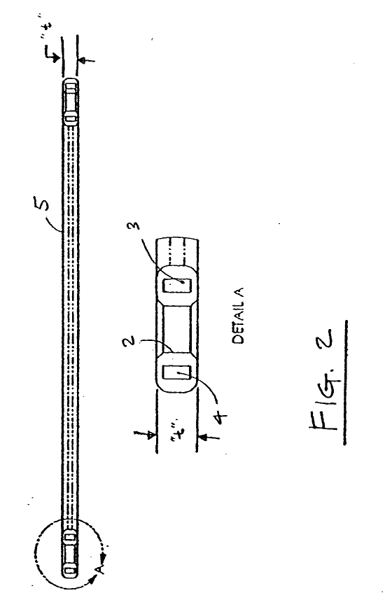

A cross-sectional view of the frame 1, as in FIG. 2, discloses a smaller stiffening ring 3 and a larger stiffening ring 4 embedded in the frame 1 annulus for the purpose of providing rigidity and durability to the frame 1. Preferably, stiffening rings 3 & 4 are comprised of radiolucent metal, such as beryllium. The diameters of the smaller ring 3 are greater than the inner diameter of the frame 1 annulus, and the diameters of the larger ring 4 are less than the outer diameter of the frame 1 annulus. Each aperture 2, as more clearly seen in FIG. 1, is situated within the area 5 defined by the smaller ring 3 and larger ring 4.

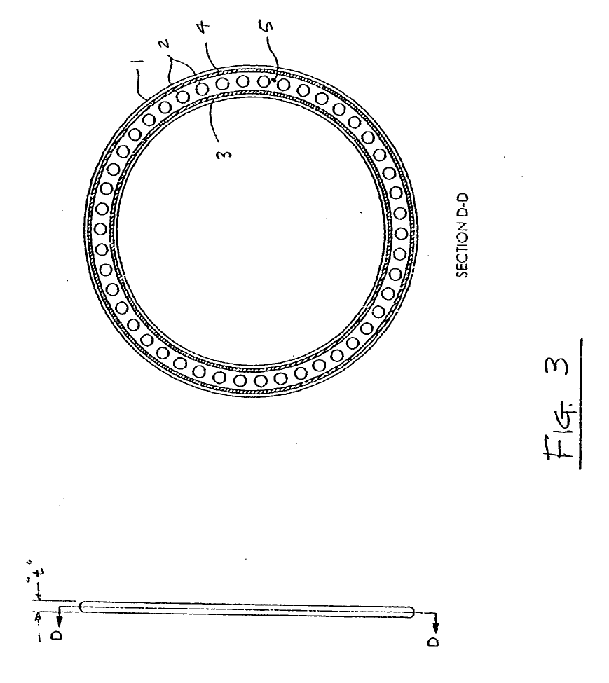

An orthogonal cross-sectional view of the frame 1, as in FIG. 3, further discloses the relative disposition of each stiffening ring 3 & 4 within the annular frame 1, as well as the relative situation of each aperture 2 with respect to the stiffening rings 3 & 4 and annular frame 1. Each stiffening ring 3 & 4 preferably forms an unbroken circle. However, those of skill in the art will appreciate that stiffening rings 3 & 4 may be arcuate.

The dimensions of the annular frame 1 may be varied so as to accommodate the physiology of the long bones to be treated, as well as the soft tissue surrounding those bones.

### Use of the Preferred Embodiment

Initially, an annular frame size is selected according to the anatomical portion upon which it will be installed, and should provide approximately 0.5-0.75 in. clearance between frame and limb. By way of illustration, as in FIG. 4, installation of the fixator upon the tibia 9 by transfixing wire 7 is described. More particularly, use of the fixator to lengthen the tibia 9 is generally described.

In most instances, two annular frames will be connected by at least three adjustable rods 6 disposed through apertures of each frame 1 such that a system of rigid alignment of one frame 1 with respect the other along a central axis (tibia 9) is achieved.

In the case of bone extension, the tibia 9 is circumferentially scored 10 to advantageously utilize the piezoelectric properties of bone. The tibia 9 is centered within the annular frame system such that one annular frame 1 is located on each side of the score 10. In the each of the regions encircled by a frame 1, two to four wires 7 transfix the bone in a manner that avoids tendons or neurovascular elements. Each wire 7 is tensioned and each wire end is secured to a wire carriage 8 attached to the frame.

Upon installation of the fixator, distraction force is introduced by adjusting the connector rods 6 to increase the distance between the annular frames 1. This pressure causes electrical charges to be generated at the score 10 site, thus focusing bone growth in that region. Over time, as the annular frames are continuously urged away from each other, the bone lengthens.

I claim:

1. A frame adapted for use with an external fixation device used in treating bone trauma and correcting deformities, said frame to at least partially encircle a bone, said frame comprising:
(a) substantially radiolucent body material; and
(b) a beryllium stiffening member
wherein said beryllium stiffening member is embedded in said substantially radiolucent body material.

2. The device of claim 1, wherein said frame is annular.

3. The device of claim 1, wherein said frame is arcuate.

4. The device of claim 1, wherein said frame is both lightweight and rigid.

5. The device of claim 1, wherein said frame is chemically inert with respect to the human body.

6. The device of claim 1, wherein said frame is chemically inert with respect to household substances.

7. The device of claim 1, wherein said substantially radiolucent body material comprises a polycarbonate compound.

8. The device of claim 1, wherein said frame is autoclavable.

9. The device of claim 1, wherein said frame is adapted for attachment of a variety of wire- and pin-securing devices.

10. The device of claim 1, wherein said frame further comprises a second beryllium stiffening member embedded in said substantially radiolucent body material.
